(19) United States
(12) Patent Application Publication
(10) Pub. No.: US 2020/0253429 A1
(43) Pub. Date: Aug. 13, 2020

(54) SYSTEM AND METHOD FOR IMPROVING HAND-HYGIENE QUALITY

(71) Applicants: Yves MARTIN, Chexbres (CH); Yves-Alain ROBERT, Rolle (CH)

(72) Inventors: Yves MARTIN, Chexbres (CH); Yves-Alain ROBERT, Rolle (CH)

(21) Appl. No.: 16/602,900

(22) PCT Filed: Jun. 25, 2018
(86) PCT No.: PCT/CH2018/000027

§ 371 Date: Apr. 16, 2020

(30) Foreign Application Priority Data
Jun. 23, 2017 (CH) 830/17

(51) Int. Cl. A47K 5/12; A61L 2/00

(52) U.S. Cl. CPC A47K 5/1217; G04F 10/00; A61L 2/0088; A47K 5/1201

(57) ABSTRACT

The invention relates to a system and method for improving the quality of hand hygiene. It comprises a portable bottle of disinfectant equipped with electronics and an electronic wristband. It gives different real-time feedback to its user regarding the quantity of disinfectant used, the duration of hand rubbing and the compliance of his/her rubbing gestures with a reference sequence. According to variations, the bottle can be used on its own.

### TECHNICAL FIELD

The present invention relates to a system and method for checking and improving the quality and disinfection or washing of carers' hands in a hospital environment.

### STATE OF THE ART

The infections associated with care in health establishments (nosocomial infections) are a major public health problem. Hundreds of millions of new episodes occur each year throughout the world, leading to millions of deaths, as well as extremely high financial loss. In Switzerland, they are responsible for 2000 deaths and additional costs of 350 million CHF each year. The main cause of this is the transmission of germs present on the hands of medical care personnel.

In order to combat nosocomial infections, the hand hygiene measures such as washing the hands or rubbing the hands with a hydro-alcoholic solution remain the most effective methods of protection. Good hand hygiene prevents 50 to 70% of these infections.

The quality of the hand hygiene of carers is determined by two parameters, “when” and “how”:

The observance of good care hygiene practices that determines whether the disinfection of hands is performed at the opportune moment based on the standards published by the World Health Organisation (WHO) is the “when”. The second parameter is based on the “how” which is the way in which the hands are disinfected. It can be expressed quantitatively by the following parameters: volume of disinfectant used during a disinfection, duration of hand rubbing once the disinfectant has been applied, compliance with a reference sequence of gestures, the microbiological effectiveness of which has been validated in the laboratory and elsewhere.

Observations conducted by hospitals of their personnel show that the rate of good practices of caregivers regarding hand hygiene is often between 20 and 50%.

Various solutions exist to improve the quality of caregivers' hand hygiene and thus reduce nosocomial infections such as, for example, training campaigns for medical care personnel or monitoring by highly qualified personnel to ensure that caregivers comply with the hand hygiene recommendations and to provide feedback on the results.

These different strategies, on average, enable an increase in compliance with hand hygiene recommendations of 20 to 50%. However, they require a significant investment of human and financial resources for a relative efficacy having a limited effect over time.

Moreover, recourse to observers introduces an “observation effect”, a bias that overestimates the true level of observance measured. The ideal would therefore be to conduct an “anonymous” observation of the caregivers. But, the means available give rise both to ethical problems (surveillance by cameras) and to technical and logistical problems associated with excessive costs.

A need therefore exists for a low-cost device and/or method that encourages and trains caregivers to adopt a good hand hygiene technique. Such a system, in addition to having a direct impact on the lives of thousands of people would provide detailed statistical analyses devoid of the bias of observation with respect to the quality of hand hygiene for healthcare establishments.

Various solutions have been proposed by the prior art. US 2007/0020212 describes a disinfecting system that activates a timer in order to encourage caregivers disinfecting their hands to rub their hands for the recommended duration. Once the set time has elapsed, the system emits a signal to alert the caregiver that the duration of hand rubbing is compliant.

US20110193703 presents a system for measuring the duration of hand disinfection performed by caregivers by means of a fluid sensor activating a wristband that sets the measurement. The users' data are made available to the system via a computer.

US2016093194 describes a system composed of an electronic wristband counting the number of gestures comprising the hand disinfection sequence. If the sequence is not completed, the system gives the wearer negative feedback so that he/she is made aware that his/her gestures are non compliant.

WO2015087331 presents a system that detects the moments when a disinfection must be performed and for a caregiver detects whether his/her hand hygiene is compliant. The user can consult his/her data on a computer, which shows whether he/she is disinfecting his/her hands at the appropriate moment.

### BRIEF SUMMARY OF THE INVENTION/DESCRIPTION OF THE INVENTION

The aim of the present invention is to propose a device and a method to enable its user to improve the quality of his/her hand hygiene by providing him/her with real-time feedback on three levels:
The quantity of disinfectant liquid used
The duration of hand rubbing performed during disinfection
The compliance of his/her movements in relation to a set sequence of gestures, of which the efficiency on a microbiological level has been tested and checked in the laboratory or complies with WHO standards.

The definition of the sequence and the measurement of its effectiveness are established as follows. Firstly the sequence of gestures to be performed is defined theoretically. Then, in the laboratory, germs (different reference strains) are deposited on the hands of the caregivers and the contamination is assessed according to various techniques, then the caregiver performs disinfection using a controlled volume of disinfectant product, for a controlled duration and according to the sequence of gestures to be tested. Finally, the contamination remaining on the hands is assessed according to different techniques. The reduction of contamination enables the effectiveness of the sequence to be assessed.

The system can also provide the user with feedback on the quality of his/her hand hygiene via a web page or mobile application.

In order to measure the parameters linked to disinfection, the invention comprises a bottle of disinfectant and a wristband.

Unlike the state of the art, the present invention proposes a system capable of providing real-time feedback on the quantity of disinfectant liquid dispensed, real-time feedback concerning the duration of rubbing and real-time feedback on the compliance of the sequence of hand rubbing gestures with a defined sequence of gestures of which the effectiveness on the microbiological level has been tested and checked in the laboratory or complies with WHO standards.

The transmission of this feedback on the quality of hand hygiene gestures places the user of the system in conditions that promote hand disinfection at the appropriate moment in compliance with WHO standards. This avoids heavy equipment, with respect to the infrastructure of a healthcare service, and aims to improve this parameter as presented in the state of the art.

This system is also characterised by the fact that it can be adapted to each user through a parameterisation connected with that individual, such as taking hand size into account for example.

The invention is composed of two elements, a portable bottle of disinfectant and an electronic wristband. The portable bottle of disinfectant equipped with an electronic unit enabling the real-time measurement of the quantity of disinfectant dispensed from the bottle of disinfectant. It is also equipped with a wireless communication circuit, a battery, a feedback system and a wireless recharging system.

The wristband is also equipped with an electronic system. This enables real-time measurement of the disinfection duration and the sequence of gestures performed during the latter. It is also equipped with a wireless communication circuit, a battery, a feedback system and a wireless recharging system. These two elements enable the following sequence to be performed, aimed at improving the hand hygiene of the wearer and their user.

During a disinfection, the caregiver begins by dispensing some disinfectant. As soon as a flow path is detected by the bottle, the latter communicates the signal corresponding to an imminent start of disinfection. During this dispensing, once the appropriate quantity of liquid is reached, feedback is provided to the user by means of a vibration (or by another way) of the bottle. The limit quantity for activating feedback is custom parameterised for each user based on hand size. In fact, the quantity of disinfectant required for good microbiological efficiency is proportional to the surface area of a person's hand (Bellissimo-Rodrigues, Soule, Gayet-Ageron, Martin, Pittet, Should Alcohol-Based Handrub Use Be Customized to Healthcare Workers' Hand Size?, Infect Control Hosp Epidemiol, 2016; 37(2):219-221). If this parameter is not entered, an average value is taken into account.

When the wristband receives the signal from the bottle corresponding to a start of disinfection, the latter starts detection of the start of hand rubbing. Once this is detected, the real-time measurement of the duration of disinfection as well as that of the sequence of gestures is activated on the basis of information transmitted by the movement sensors. The detection of contact between the hands of the wearer of the system can also assist in the measurement if the electronics of the wristband so permits.

During the measurement, the wristband can receive information relating to the quantity of disinfectant dispensed by the user and this data can be used to parameterise the duration of hand rubbing required to activate the wristband's feedback. This duration can also be individual and fixed. If this duration is not entered, it is set at an average value.

Once the parameterised rubbing duration has been reached, the wristband provides feedback to its wearer in the form of a vibration for example. During the disinfection gestures, the wristband sends to the user, in the form of a vibration for example, information relating to the quality of his/her gestures if compliance with the reference sequence is confirmed.

If the user fails to achieve the set goals, the corresponding feedback is not given.

Feedback concerning the duration of disinfection can also be parameterised at a fixed duration: the vibration (or other signal) will occur after the fixed duration even if the caregiver rubbed his/her hands for a shorter period of time. The caregiver knows that he/she must disinfect his/her hands for the fixed duration.

This real-time feedback allows the user to:

a) put an adequate quantity of disinfectant on his/her hands in order to achieve sufficient effective disinfection based on hand size;

b) perform hand disinfection rubbing for a duration that allows good disinfection effectiveness, regardless of the quantity of disinfectant and hand size;

c) perform rubbing with gestures whose microbiological effectiveness has been validated in the laboratory or any other recommended gestures.

The battery of the bottle and that of the wristband can be charged with or without contact.

The bottle and wristband record the data linked to each disinfection measurement and send them to a database directly by Wi-Fi or via a connection to a relay device.

Any other element that enables the measurement and recording of the quantity of disinfectant dispensed and the duration of rubbing and disinfection gestures and that can give feedback and communicate the measurement data to a server, whether via an external element or not, can be used to perform the sequence as described.

Advantageously, in addition to enabling the user of the system to improve his/her hand hygiene, the data recorded (date, time, quantity of disinfectant, rubbing duration, rubbing gestures) allow the user to access a summary of the quality of his/her hand hygiene via a web page or mobile application. Moreover, these data can be used for study purposes in healthcare establishments.

Advantageously, the system can thus be used in an existing hospital for a minimum installation cost, without infrastructure and on a modest scale, for example with just one user or one dispensing point.

### BRIEF DESCRIPTION OF THE FIGURES/LIST OF DRAWINGS

Examples of implementation of the invention are indicated in the description illustrated by the accompanying Figures, in which:

FIG. 1 shows a schematic view of the elements required to perform the hand hygiene improvement and monitoring sequence. In this version, the bottle is coupled with a wristband;

FIG. 2 shows a schematic view of a bottle according to the invention;

FIG. 3 shows a schematic view of a wristband according to the invention;

FIG. 4 shows the block diagram of a hand hygiene improvement and monitoring sequence.

### DETAILED DESCRIPTION OF THE INVENTION

The system of the present invention is designed to be implemented in any field where hand hygiene is important, such as the hospital environment for example.

In the present application, the word “bottle” must be interpreted broadly so as to include any type of portable liquid container.

In the present application, the words “disinfection” and “hand washing” must be interpreted broadly so as to include any operation during which a user uses a bottle to dispense disinfectant material and rubs his hands with it.

In the present application, the words “liquid”, “liquid disinfectant” and “disinfectant” must be interpreted broadly so as to include any substance enabling hand washing or disinfection dispensed from a bottle and of which the volume used during one dispensing operation is measurable.

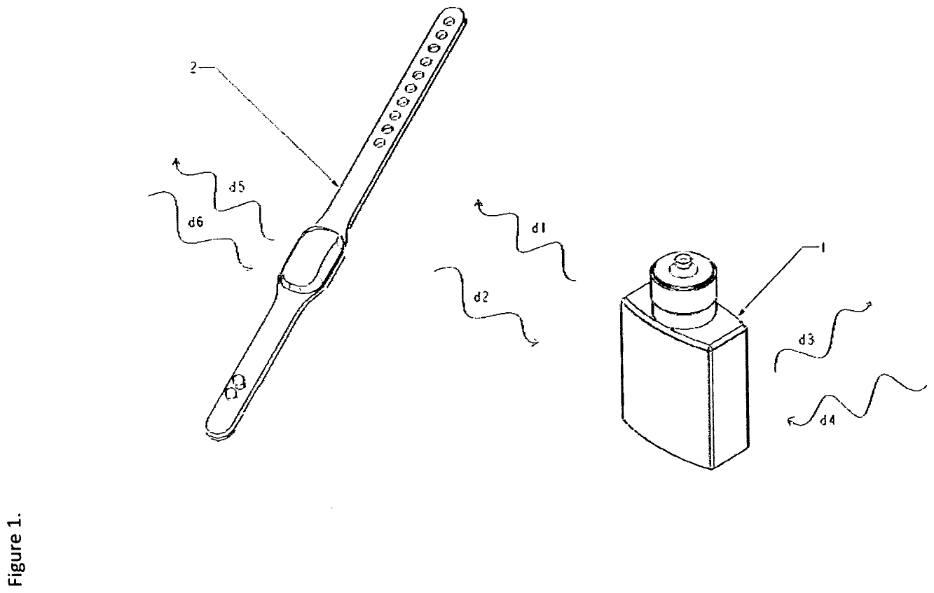

The system shown schematically in FIG. 1 comprises two elements, a bottle 1 and a wristband 2, which provide their user with real-time feedback on the quality of his/her hand hygiene and thus offer him/her the possibility to improve it. They record the data relating to the hand disinfections performed and send them to a database.

Bottle 1 enables real-time measurement of the volume of liquid dispensed from the bottle.

The wristband 2 enables real-time measurement of the duration of hand rubbing following a dispensing of disinfectant as well as the real-time assessment of the compliance of the rubbing gesture with a reference sequence, the microbiological effectiveness of which has ben tested and verified in the laboratory or complies with WHO standards.

The bottle 1 and the wristband can give feedback individually, for example by means of a vibration, and both contain a wireless communication element enabling an exchange of data between them or with an external element. The data d1 and d2 correspond to the different signals exchanged between the wristband and the cap such as a signal corresponding to the start of a disinfection for example. Data d3 and d5 correspond to the recording of measurements made by the bottle and the wristband respectively relating to the disinfections and their status in an external database. Data d4 and d6 correspond to the receipt of measurement parameters for the bottle and the wristband respectively when this information is present in an external system such as a server for example. These parameters are the quantity of reference disinfectant for the user of the system as well as the reference duration of disinfection for the latter.

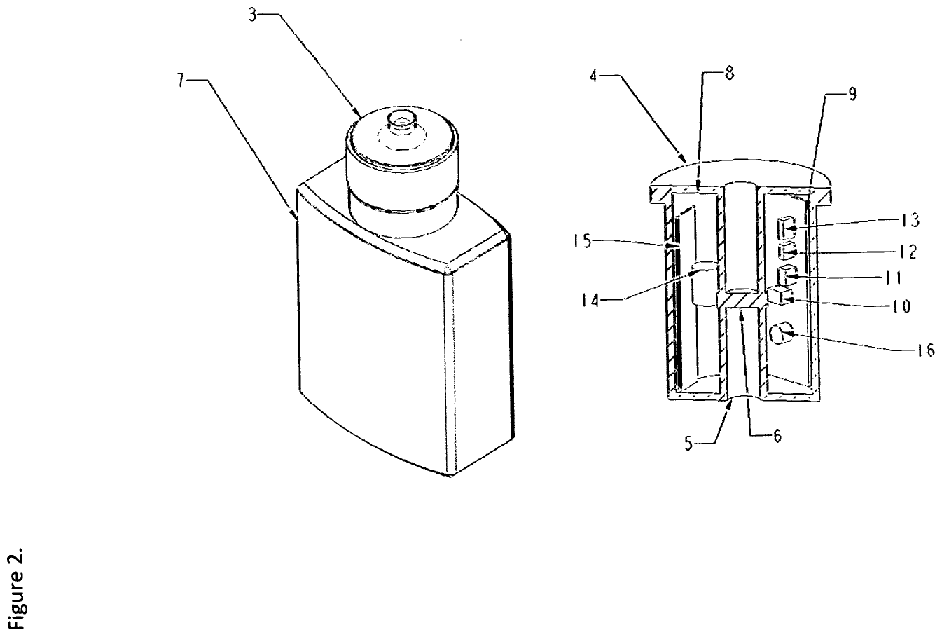

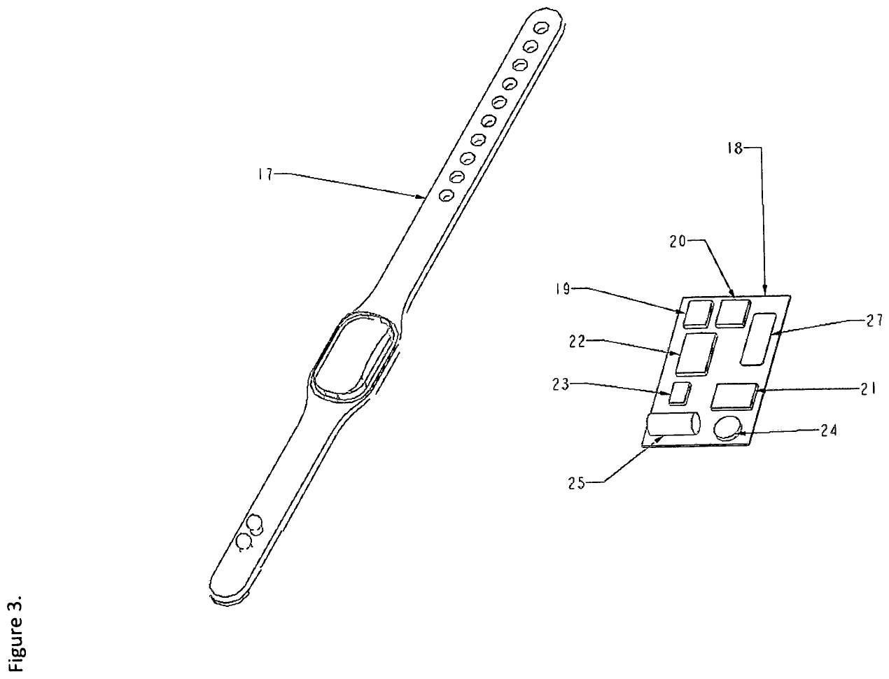

Bottle 1 and wristband 2 will described in more detail in relation to FIGS. 2 and 3 respectively.

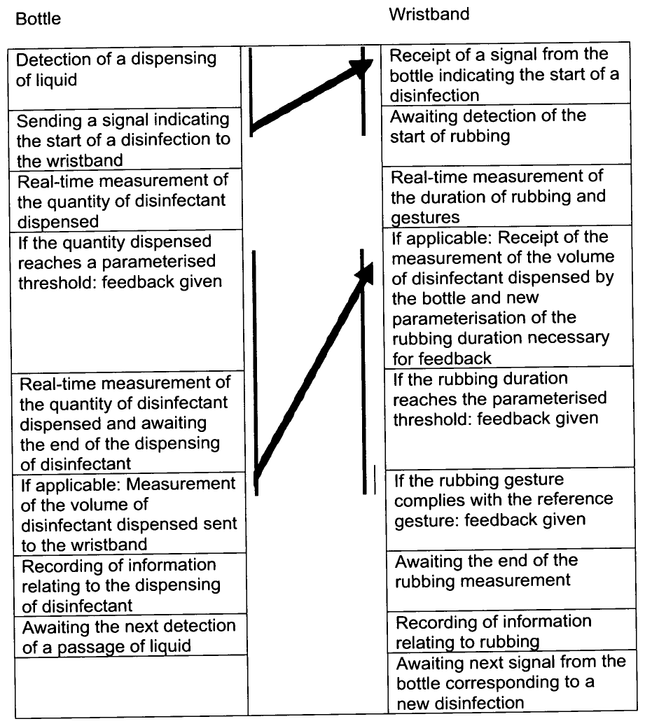

Any other element that can measure the quantity of disinfectant, the hand rubbing duration and the compliance with a sequence of gestures in real time as well as being able to give feedback and communicate directly or indirectly with a server can be used to perform the sequence according to the invention described in greater detail in relation to FIG. 4.

FIG. 2 shows a schematic representation of the bottle 7 corresponding to the bottle 1 in FIG. 1. It comprises a closing system 3 fixing onto the neck. Inside the neck a cylinder 4 is placed containing a flow duct 5 along which a flow meter 6 is placed.

The cylinder 4 contains a sealed space 8 between its walls and the flow duct 5. In the latter is placed an electronic circuit comprising a flow sensor 10, a processor 11, a memory 12, a wireless communication element 13 and a system 16 for giving feedback to the user. The electronic circuit 9 is powered by a battery 14, rechargeable by induction by means of the coil 15.

The flow sensor 10 enables a use of the bottle to be detected and the volume of liquid dispensed to be measured in real time.

The feedback element 16 can, for example, be a vibrator, a LED, a loudspeaker or any other type of equipment enabling a sensory signal to be transmitted.

The wireless communication element 13 can, for example, be a Bluetooth, NFC (Near Field Communication), Wi-Fi or proprietary interface.

FIG. 3 schematically shows the wristband 17 corresponding to the wristband 2 in FIG. 1. It contains an electronic circuit 18 comprising an accelerometer 19, a gyroscope 20, a processor 21, a memory 22, a wireless communication element 23 and an element to send feedback to the user 24. The electronic circuit 19 is powered by a battery 25 rechargeable by induction by means of the coil 26.

The accelerometer 19 and the gyroscope 20 make it possible to measure the duration of rubbing during a disinfection of the hands performed by the wearer of the wristband as well as to assess the gestures in relation to a reference sequence.

The feedback element 24 can for example be a vibrator, a LED, a loudspeaker or any other type of equipment enabling the transmission of a sensory signal. The wireless communication element 23 can, for example, be a Bluetooth, NFC (Near Field Communication), Wi-Fi or proprietary interface. This interface is identical to that of the wireless communication element 13 present in the bottle 7.

FIG. 4 is a schematic representation of the sequence of measuring the hand hygiene quality of the wearer of the system and of providing feedback.

The disinfection sequence is initiated when the wearer of the system dispenses disinfectant from his bottle. As soon as the use of the bottle is detected, the bottle sends a signal to the wristband indicating a start of disinfection while measuring the volume of disinfectant dispensed in real time. During the measurement of the dispensed volume, if the latter exceeds a threshold value previously parameterised and capable of being linked to the user's hand size, a feedback signal comprising a vibration, for example, is sent to the user. It is also possible at that point to send a signal to the wristband so that the feedback is not given by the bottle but by the wristband.

When the disinfection dispensing operation is completed, the data linked to this operation are recorded in the bottle's memory. The bottle can also send these data directly to the wristband so that they are recorded in the memory thereof or so that the quantity of dispensed disinfectant is used in order to parameterise the hand rubbing duration required for an activation of feedback.

After this, the bottle again awaits the next detection of dispensing disinfectant. When the wristband receives the signal of the start of disinfection from the bottle, the latter awaits detection of the start of hand rubbing. Once this is detected, the measurement of the duration of this rubbing is started, together with the comparison of the gestures to the reference sequence based on the accelerometer and the gyroscope contained therein.

If the bottle transmits information on the quantity of disinfectant used to the wristband, the latter can adjust the minimum time threshold value to be reached in order to activate feedback. In the opposite case, the threshold value can be parameterised manually through a web page or mobile application or is even defined by default.

When measuring the duration of rubbing, if it exceeds the threshold value, feedback is given to the user in the form of vibration for example by the wristband to alert its wearer that the recommended duration has been reached.

When measuring rubbing, if compliance of the sequence of gestures with the reference sequence is detected, the wristband sends feedback to alert its wearer that this is the case.

When completion of rubbing is detected, the information linked to the measurement of the duration of rubbing as well as to the gestures performed is recorded in the wristband's memory.

1. Method and device for measuring and improving the washing of hands characterised by:
a portable bottle (1) containing a liquid and capable of measuring the volume thereof in real time and of sending feedback to the user;
an electronic wristband (2) provided with sensors required to measure the gestures of its wearer and capable of transmitting feedback to the user;
a sequence performed during a hand disinfection by the user of said portable bottle (1) and electronic wristband (2) in order to enable the caregiver to improve his/her hand hygiene:
a) real-time measurement of the quantity of liquid by means of said portable bottle (1);
b) once the quantity of liquid dispensed corresponds to the reference quantity, transmission of feedback;
c) real-time measurement of the duration of hand rubbing by means of said electronic wristband (2);
d) once the hand rubbing duration corresponds to the reference duration, transmission of feedback.

2. Method according to claim 1 wherein a real-time measurement of the compliance of the sequence of hand washing gestures with a defined reference sequence of gestures of which the efficiency on a microbiological level has been tested and checked in the laboratory or complies with WHO standards, or other standards, can be performed by means of said electronic wristband (2).

3. Method according to claim 2 wherein real-time feedback can be transmitted to the user of said electronic wristband (2) when the sequence of gestures performed complies with said reference sequence of gestures.

4. Device according to claims 1 to 3 wherein said real-time feedback can be given by a vibration or any other sensory signal by said bottle (1), said electronic wristband (2) or any other connected object.

5. Device according to claim 1 wherein an interaction between the data measured by said bottle (1) and said electronic wristband (2) is possible in order to alter the reference duration of hand rubbing so as to activate the feedback relating thereto.

6. Device and method according to claims 1 to 5 wherein the feedback can be deactivated or given after disinfection.

7. Device according to claim 1 wherein determining contact between the hands contributes towards determining the duration of disinfection.

8. Device according to claims 1 and 8 wherein said electronic wristband (2) can be provided with a device, for example an aerial, that can be activated before the start of a disinfection gesture and whose signals enable measurement of the contact of the hands of the system's user.

9. Device according to claims 1 and 8 wherein said inertial sensors (20, 21) of said electronic wristband (2) can detect the characteristic data at the moment when both hands come into contact and then when they separate.

10. Device according to claim 1 wherein said bottle (1) can contain an inertial sensor the data of which teach the manner of dispensing liquid for each user and thus enable said bottle to be calibrated so as to enable a measurement of the volume of liquid dispensed based on its data.

11. Device according to claim 1 wherein said portable bottle (1) can comprise a valve, activated by the processor (11), enabling the volume of disinfectant dispensed to be blocked at a parameterised value.

12. Device and method according to claims 1 to 11 wherein the feedback transmitted improves observance of the hand hygiene of the wearer of the system.

13. Method and device for measuring and improving hand washing characterised by:
a portable bottle (1) containing a liquid and capable of measuring the volume thereof in real time and of transmitting feedback to the user;
a sequence performed during a hand disinfection by the user of said portable bottle (1) in order to enable the caregiver to improve his/her hand hygiene:
a) real-time measurement of the quantity of liquid by means of said portable bottle (1);
b) once the quantity of liquid dispensed corresponds to the reference quantity, transmission of feedback.

14. Device according to claim 13 wherein said real-time feedback can be given by a vibration or any other sensory signal by said bottle (1) or any other connected object.

15. Device according to claim 13 where said bottle (1) can contain an inertial sensor the data of which teach the manner of dispensing liquid for each user and thus enable said bottle to be calibrated so as to enable a measurement of the volume of liquid dispensed based on its data.

16. Device according to claim 13 wherein said portable bottle (1) can detect the surface of the user's hand by means of an element connected to said electronic system.

17. Device according to claim 13 wherein said portable bottle (1) obtains the surface of the user's hand by wireless communication with an external system worn by the caregiver.

18. Device according to claim 13 wherein said portable bottle (1) obtains the surface of the user's hand from a database by means of a user identifier obtained by wireless communication with an external system worn by the caregiver.

19. Device and method according to claims 13 to 18 where the feedback can be deactivated or given after disinfection.

20. Device according to claim 13 wherein said portable bottle (1) can comprise a valve, activated by the processor (11), enabling the volume of disinfectant dispensed to be blocked at a parameterised value.

21. Device according to claim 13 wherein said portable bottle (1) can be arranged on a fixed support and where a target volume of disinfectant solution can be dispensed automatically by said portable bottle equipped with a pump operated by said electronic system.

22. Device according to claim 13 wherein said portable bottle (1) can be arranged on a fixed support and where the liquid is dispensed automatically by said portable bottle equipped with a pump operated by said electronic system. This being the case for as long as the user has one (or both) hands, detected by, beneath the device. Once the quantity is reached, the user receives feedback telling him/her to withdraw his hand(s).

23. Device and method according to claims 13 to 22 wherein the transmitted feedback enables observance to hand hygiene by the user of the portable bottle to be improved.
